(19) United States
(12) Patent Application Publication
(10) Pub. No.: US 2025/0009831 A1
(43) Pub. Date: Jan. 9, 2025

(54) COMBINATION OF SCUTELLARIA EXTRACTS WITH CITRUS BERGAMIA OR CYNARA CARDUNCULUS EXTRACTS AND FORMULATIONS CONTAINING THEM

(71) Applicant: AKHYNEX S.R.L., Polistena (RC) (IT)

(72) Inventors: Ezio Bombardelli, Gropello Cairoli (PV) (IT); Elena Lombardo, Bianco (RC) (IT)

(73) Assignee: AKHYNEX S.R.L., Polistena (RC) (IT)

(21) Appl. No.: 18/262,924

(22) PCT Filed: Jan. 28, 2022
(86) PCT No.: PCT/IB2022/050745

§ 371 Date: Jul. 26, 2023

(30) Foreign Application Priority Data
Feb. 1, 2021 (IT) 102021000001994

(51) Int. Cl. A61K 36/539; A61K 36/28; A61K 36/752; A61K 45/06

(52) U.S. Cl. CPC A61K 36/539; A61K 36/28; A61K 36/752; A61K 45/06

(57) ABSTRACT

Disclosed is a combination of Scutellaria extracts with Citrus bergamia or Cynara cardunculus extracts, which is useful for the prevention and treatment of fatty liver disease.

The present invention relates to a combination of Scutellaria baicalensis extracts with Citrus bergamia or Cynara cardunculus extracts, which is useful for the prevention and treatment of fatty liver disease.

### PRIOR ART

Non-alcoholic fatty liver disease (NAFLD) is one of the most frequent causes of liver damage, often associated with cirrhotic degeneration and hepatocellular carcinoma. The origin of NAFLD is not yet fully understood. Many studies have demonstrated that some inflammatory markers, such as TNF-α and other cytokines, play an important part in its pathogenesis. Additional factors are obesity and metabolic disorders associated with insulin resistance. No medicaments are yet available, and the only measures currently recommended for individuals suffering from NAFLD are lifestyle changes, attention to diet, and weight loss.

Scutellaria baicalensis is a plant widely used in Chinese medicine. In particular, its root extracts have been used in the treatment of inflammatory states and respiratory problems of allergic origin, and to support the body's physiological reaction and defense mechanisms. Scutellaria baicalensis has antibacterial, antiviral, antioxidant, hepatoprotective and neuroprotective properties. Flavonoids such as baicalin, baicalein, wogonin and skullcap flavone are believed to be the main active ingredients responsible for the activities observed in rats treated with orotic acid to induce fatty liver disease; baicalin inhibited the accumulation of fats in the liver, attenuated hypertrophy of the hepatocytes, and had a preventive effect on FFA-induced lipotoxicity (Qian Chen et al., J NatMed 2018 June; 72(3):655-666).

The bergamot orange (Citrus bergamia Risso & Poiteau), a citrus fruit grown in small areas of Calabria, Italy, has mainly been used for the properties of its essential oil, which is particularly requested in perfumery.

Bergamot orange juice, which is rich in polyphenols and flavonoids (brutieridin and melitidin (Mollace et al., Fitoterapia, vol. 82, no 3, 2011, pp. 309-316)), also possesses cholesterol-lowering and blood glucose-lowering activity.

U.S. Pat. No. 8,741,362 describes a plant extract obtained from the pith and juice of the fresh fruit of Citrus aurantium var bergamia, which normalises the lipid and blood glucose parameters in patients suffering from diet-dependent or inherited metabolic disorders. In pathological hyperlipaemia, a reduction in total cholesterol and LDL cholesterol has been demonstrated at concentrations ranging between 20 and 32%, with a 30% increase in HDL cholesterol. These major variations are associated with a marked reduction in VLDL cholesterol and a dimensional increase in LDLs, leading to a reduction in their oxidation. Bergamot orange extract has also improved the endothelial function, with vascular protection attributable to flavonoids. In a fatty liver disease model in transgenic mice developing a condition similar to the human disease, bergamot orange extract significantly counteracted perisinusoidal fibrosis, which is the most difficult condition to modify in NAFLD.

The choleretic, cholagogic, hypoglycaemic, anti-dyspepsia and hypocholesterolaemic action of Cynara cardunculus var sylvestris extracts is also known.

Clinical trials report modest, erratic increases in HDL cholesterol following administration of high doses of Cynara cardunculus extracts, amounting to several grams a day (Naturmed, 13, 17-24,1998; ArzneimForschung, 50, 260-65, 2000; The Cochrane Library, 2002, Issue 3). Standardised artichoke extracts containing 25% by weight of caffeoylquinic acids, 8% by weight of flavonoids and 7% by weight of cynaropicrin, which possess improved hypolipidaemic and hypoglycaemic activities, have been developed in recent years (Rondanelli et al., Phytotherapy Research 28:33-41 (2014) and Italian Journal of Medicine 2014; Vol 8, suppl. 2, p. 113). Said extracts only exhibit a reasonable level of activity if administered twice a day, and in any event have little effect on major hyperlipidaemia.

The choleretic, hypoglycaemic and liver-protecting activity of artichoke extracts is attributed to caffeoylquinic acids, while flavonoids perform a hypolipidaemic action associated with cholesterol synthesis, and cynaropicrin exerts an anti-inflammatory and anti-STAT 3 action. Cynara scolymus extracts deprived of cynaropicrin, prepared from non-bitter flower heads, have no effect on cholesterol, only on blood glucose. In a recent study, Cynara cardunculus var sylvestris extracts with a high cynaropicrin content were tested on NAFLD in mice, providing significant protection against fatty liver disease induced by a high-fat, high-glucose diet (Oppedisano et al., Nutrients 2020, 12(5), 1435).

### DESCRIPTION OF THE INVENTION

It has now been found that the flavonoids present in plants belonging to the genus Scutellaria, such as baicalin, baicalein, wogonin and skullcap flavone, combined with extracts of Citrus aurantium var bergamia (bergamot orange) or Cynara cardunculus var sylvestris having a high cynaropicrin content, give rise to a significant improvement in the lipid and carbohydrate parameters and a much greater reduction in non-alcoholic fatty liver disease than is obtained by administering the individual extracts, even at higher doses.

The subject of the invention is therefore compositions comprising Scutellaria baicalensis flavonoids or extracts containing them, and an extract selected from Cynara cardunculus extract and Citrus aurantium var bergamia extract.

A first aspect of the invention relates to compositions containing Scutellaria baicalensis extract and Citrus aurantium var bergamia extract.

In particular, non-purified technical juice is used, obtained by pressing the crushed fruit under controlled temperature conditions, followed by centrifugation, clarification and pasteurisation of the juice, and concentration until dry. Scutellaria baicalensis extract, Cynara cardunculus var sylvestris extract, or both, can be added to the juice before final concentration. The pressing residue, rich in soluble and insoluble fibres and pectins, is dried with a spray-dryer or a vacuum unit and, after addition of Scutellaria baicalensis extract, is micronised and can be used unmodified to prepare functional foods, in particular in the form of bars.

A second aspect of the invention relates to compositions containing extract of Scutellaria baicalensis and extract of the sylvestris variety of Cynara cardunculus.

In the case of the combination of Citrus bergamia, Scutellaria baicalensis and also Cynara cardunculus var sylvestris extracts, it has proved advantageous to dissolve the dried extracts in water-alcohol mixtures until complete dissolution of the ingredients, to obtain a composite extract which, after concentration until dry, exhibits a good degree of water-solubility and a synergic increase in the properties of the individual extracts, resulting in unexpected preventive and therapeutic activity against fatty liver disease at much lower doses than those of the individual extracts.

In the case of the combination of Scutellaria baicalensis extract and Cynara cardunculus var sylvestris extract, for reasons of reciprocal solubility, it has proved advantageous to dissolve the two extracts in ethyl acetate in the presence of an equal weight of saturated, vacuum-concentrated phospholipids until complete elimination of the solvent.

The invention therefore also relates to composite extracts obtained by complete solubilisation and subsequent drying of a mixture of a Scutellaria baicalensis extract and a Citrus aurantium var bergamia extract. The invention also relates to composite extracts obtained by complete solubilisation and subsequent drying of a mixture of a Scutellaria baicalensis extract and a Cynara cardunculus var sylvestris extract in the presence of phospholipids.

The invention further relates to said compositions for use in the treatment and prevention of fatty liver disease and the treatment of dyslipidaemia, metabolic syndrome, type 2 diabetes and cardiovascular disease.

### DETAILED DESCRIPTION OF THE INVENTION

The Scutellaria baicalensis extract usable according to the invention typically comprises 85% baicalin and 6% baicalein.

A preferred extract of Citrus aurantium var bergamia is obtained by pressing the crushed fruit cold at temperatures ranging between 0 and 4° C., and at pressures ranging between 200 and 300 bars, preferably 250 bars, clarifying the juice with a decanter and pasteurising the juice. The juice can be preserved unmodified under sterile conditions and used by adding the other extracts, or concentrated and atomised. The extract contains about 25% total flavonoids, 19% of which consists of naringin, 18.6% of neohesperidin, 15.3% of neoeriocitrin, and the remainder of a fraction of flavonoids with structures similar to those of brutieridin and melitidin. The pressing residue of the fruit, still containing a significant amount of polyphenols present in the juice, and polymethoxylated flavonoids such as nobiletin, tangeretin and terpene substances, can be combined with Scutellaria baicalensis and/or Cynara cardunculus var sylvestris extracts.

The Cynara cardunculus var sylvestris extract is prepared by using the aerial parts of the plant, dried in a rotary dryer at high temperature to preserve the active ingredients unchanged, and subsequently extracting the biomass with ethanol/water mixtures; after removal of the ethanol, the clear, filtered aqueous solution is treated mainly with SEPABEADS SP absorption resin or other polystyrene resins; the resin, which retains the polyphenol substances and cynaropicrin, is washed thoroughly with water to remove inert substances, and then eluted with ethanol or an ethanol/water mixture. The eluate is concentrated by evaporating the ethanol.

The compositions according to the invention contain 10 to 300 mg of Scutellaria spp flavonoids and 50 to 300 mg of Citrus aurantium var bergamia extract or Cynara cardunculus var sylvestris extract. 150 mg to 200 mg of the two extracts is preferably used.

If soluble compositions are to be obtained, the Scutellaria baicalensis and bergamot orange extracts can be dissolved in 15 volumes of a 7:3 ethanol/water mixture, and then concentrated under vacuum. Alternatively, in the case of the combination with Cynara cardunculus var sylvestris extract, the Scutellaria baicalensis extract is suspended in 15 volumes of ethyl acetate at temperatures ranging between 3° and 60° C., preferably 40° C., in the presence of hydrogenated sunflower phospholipids under stirring until completely solubilised. An equal weight of Cynara cardunculus var sylvestris extract, having a cynaropicrin content ranging between 20 and 40%, preferably 30% by weight, is added to said solution under stirring.

The composite extract(s) is/are formulated in forms suitable for oral administration, for example as conventional or gastroprotected capsules or tablets, sugar-coated pills, soft or hard gelatin capsules, or cellulose capsules. The compositions according to the invention will be formulated by conventional methods, such as those described in “Remington's Pharmaceutical Handbook”, Mack Publishing Co., N.Y., USA. In particular, the compositions according to the invention will be formulated according to conventional plant ingredient formulation techniques, which require particular care to be taken to avoid interactions with the excipients and the capsule matrices.

Particularly suitable carriers are oils rich in ω-3 fatty acids, which facilitate the absorption of cynaropicrin and flavonoids.

The formulations can also contain other active ingredients with complementary or otherwise useful activities, in particular one or more extracts of Olea europaea, Berberis aristata, Vitis vinifera, Cyclanthera pedata, Silybum marianum and/or carotenoids.

Particularly preferred is the combination with Olea europaea extract containing 20% oleuropein and isomers thereof, verbascoside, pentacyclic terpenes and oleocanthal, compounds having potent anti-inflammatory activity which is useful in the liver in steatosis degeneration processes.

The combination with Olea europaea extract therefore maintains therapeutic efficacy in the treatment of metabolic syndrome, even at low doses of the formulations according to the invention. Said combination, in addition to the primary activity against fatty liver disease and lipid metabolism, also acts on adipose tissue and vascular inflammation. The growing adipose tissue is infiltrated by macrophages that release proinflammatory cytokines and inflammation mediators such as COX-2 and iNOS.

The dose of the extracts according to the invention typically ranges between 100 and 400 mg/day, preferably 300 mg/day, a dosage interval which is much smaller than those reported for the uncombined extracts (1.3 g for bergamot orange extract, 500 mg/day for Cynara cardunculus var sylvestris extract, and 300-500 mg for Scutellaria baicalensis extract).

The activity of the extract according to the invention was evaluated in a model of mice prone to induced fatty liver disease which, when fed on a high-lipid, high-carbohydrate diet, express obesity, insulin resistance and significant dyslipidaemia; to accelerate the timing, they can be treated with orotic acid in the diet. The organ weights and plasma lipid levels were measured at time 0 and after 4 weeks, and measured again a further 4 weeks after administration of the active ingredients. The data are set out in Tables 1 and 2.

Table 1 compares the data obtained by administering the compounds individually at the doses reported in the literature in similar experiments (dried Citrus bergamia juice 50 mg/Kg, Scutellaria baicalensis extract 50 mg/Kg), whereas in the combination, the doses are halved, so that the same amount by weight can be administered to the animals. The mixture exhibited a surprising effect.

TABLE 1
Effect of dried technical juice of bergamot orange and dried technical
juice of Scutellaria baicalensis, and of the combination
thereof according to the invention.

| Parameters | Normal | Control | STBD | SCB | STBD/SCG |
|---|---|---|---|---|---|
| Liver weight | 4.1 ± 0.1* | 8.3 ± 0.4** | 7.2 ± 02 | 6.7 ± 0.3 | 4.6 ± 0.1** |
| TG (mg/Kg) | 12.7 ± 1.1* | 116.2 ± 9.4** | 42.02 ± 11.3 | 31.4 ± 9.2 | 9.7 ± 2.2** |
| TC (mg/Kg) | 0.9 ± 0.1** | 3.6 ± 0.4* | 1.2 ± 0.6* | 1.1 ± 0.1 | 0.8 ± 00* |
| FFA (10⁻³ mEq/g) | 5.1 ± 0.2* | 15.6 ± 0.6* | 11.1 ± 0.5* | 7.7 ± 0.3 | 6.6 ± 0.6** |

STBD: dried technical juice of bergamot orange
SCB: Scutellaria baicalensis extract

TABLE 2

| Parameters | Normal | Control | CCS | SCB | CCS/SCB |
|---|---|---|---|---|---|
| Liver weight | 4.1 ± 0.1* | 8.3 ± 0.4** | 7.2 ± 02 | 6.7 ± 0.3 | 4.2 ± 0.1** |
| TG (mg/Kg) | 12.7 ± 1.1* | 116.2 ± 9.4** | 32.02 ± 10.0 | 31.4 ± 9.2 | 9.7 ± 2.2** |
| TC (mg/Kg) | 0.9 ± 0.1** | 3.6 ± 0.4* | 1.0 ± 0.6* | 1.1 ± 0.1 | 0.8 ± 00* |
| FFA (10⁻³ mEq/g) | 5.1 ± 0.2* | 15.6 ± 0.6* | 10.1 ± 0.5* | 7.7 ± 0.3 | 5.9 ± 0.2** |

CCS: Cynara cardunculus var sylvestris extract
SCB: Scutellaria baicalensis extract

A clinical trial was conducted by administering the extracts at the dose of 300 mg once a day in soft gelatin capsules. The study involved a total of 102 adult patients with fatty liver disease identified by TE (Transient Elastography), and treated with placebo (STBD/SCB combination) or STBD at the dose of 650 mg twice a day (Nutrients 2020 May 21; 12(5):1504). Patients presenting alcohol abuse, patients with signs of chronic hepatitis B or C, or liver or systemic disorders requiring specific treatments, were excluded from the trial. Fatty liver disease was evaluated with TE and CAP (controlled attenuation parameters) at three levels in the diagnosis according to severity, namely 216 and 252 dB/m respectively for the grade S1 diagnosis, 253 and 296 dB/m for S2, and CAP exceeding 296 dB/m for S3, which is the most severe (23, 24). Total cholesterol, lipoproteins, triglycerides, insulin and liver enzymes ALT, AST and GGT were evaluated. The value of 268.6±52 was taken as CAP in the evaluation, according to the procedures reported in the literature. The results of the trial are set out in Table 3 below.

TABLE 3
Baseline and 12-week follow-up values for placebo, STBD and STBD/SCB.

| | Placebo | | STBD | | STBD/SCB | | p |
|---|---|---|---|---|---|---|---|
| Parameters | Baseline | Follow-up | Baseline | Follow-up | Baseline | Follow-up | value |
| Body weight | 79 ± 11 | 77 ± 12 | 80 ± 10 | 78 ± 13 | 80 ± 11 | 76 ± 13 | <0.001 |
| BMI (Kg/m²) | 28.4 ± 3 | 27.9 ± 4 | 29 ± 3 | 28.1 ± 4 | 29.6 ± 3 | 27.5 ± 4 | <0.001 |
| CAP scores | 283 ± 28 | 270 ± 45 | 275 ± 31 | 262 ± 26 | 295 ± 35 | 247 ± 29 | <0.001 |
| (sb/m) | | | | | | | |
| TC (mg/dL) | 194 ± 37 | 189 ± 42 | 201 ± 40 | 190 ± 32 | 189 ± 33 | 175 ± 20 | <0.06 |
| TG(mg/dL) | 113 ± 39 | 110 ± 51 | 117 ± 24 | 109 ± 26 | 107 ± 14 | 102 ± 36 | <0.82 |
| LDL-C (mg/dL) | 122 ± 15 | 117 ± 35 | 115 ± 24 | 109 ± 14 | 120 ± 31 | 107 ± 26 | <0.09 |
| ALT (IU/L) | 28 ± 21 | 22 ± 9 | 26 ± 10 | 22 ± 11 | 24 ± 12 | 19 ± 7 | <0.01 |
| YGT(IU/L) | 27 ± 21 | 25 ± 22 | 26 ± 20 | 24 ± 16 | 26 ± 11 | 22 ± 14 | <0.01 |

The data indicate that the products according to the invention markedly reduce fatty liver disease compared with the placebo. The effect was even more marked in sub-groups of patients with android obesity and age exceeding 50 years. As demonstrated in other trials, the mixture of the products examined is markedly superior to the individual ingredients, even in humans.

The formulations according to the invention also proved effective on various parameters in patients suffering from metabolic syndrome, in whom normalisation of parameters such as blood glucose, lipid parameters, hypertension and “silent inflammation” was observed.

The following examples further illustrate the invention.

### Example 1—Preparation of Hard Gelatin Capsules Containing Scutellaria baicalensis Extracts and Cynara cardunculus Var Sylvestris Extract

### Preparation of 300 mg Hard Gelatin Capsules

### Unit Composition:

Scutellaria baicalensis 200 mg
Cynara cardunculus var sylvestris 100 mg
Microcrystalline cellulose 100 mg
Silicon dioxide 5 mg
Magnesium stearate 5 mg

### Example 2—Formulation of the Combination of Citrus bergamia Extracts and Scutellaria baicalensis Extracts as an Oily Suspension for Soft Gelatin Capsules

### Unit Composition

Citrus bergamia 200 mg
Scutellaria baicalensis 100 mg
Soya lecithin 200 mg
Beeswax 5 mg
Linseed oil 225 mg

### Example 3—Formulation of the Combination of Citrus bergamia Extracts and Scutellaria baicalensis Extracts as an Oily Suspension for Soft Gelatin Capsules

### Unit Composition

Citrus bergamia 150 mg
Scutellaria baicalensis 100 mg
Cynara cardunculus var sylvestris 100 mg
Beeswax 5 mg
Linseed oil 225 mg

### Example 4—Formulation of the Combination of Citrus bergamia Extracts and Scutellaria baicalensis Extracts as Chewable Bars

### Unit Composition

Citrus bergamia, dried residue 4000 mg
Scutellaria baicalensis 100 mg
Phosphorylserine 150 mg
Vitamin B6 25 mg
Acetylglutamine 100 mg
Glucomannan 3 g
Calcium caseinate 3 g
Puffed rice 5 g
Orange flavouring with dark chocolate coating.

1. Compositions comprising flavonoids of Scutellaria baicalensis plant or extracts containing them, and an extract selected from Cynara cardunculus var sylvestris extract and Citrus aurantium var bergamia extract.

2. Compositions according to claim 1 wherein the flavonoids are baicalin, baicalein, wogonin and skullcap flavone.

3. Compositions according to claim 1 comprising a Scutellaria baicalensis extract.

4. Compositions according to claim 1, comprising a Scutellaria baicalensis extract and a Citrus aurantium var bergamia extract.

5. Compositions according to claim 1, comprising a Scutellaria baicalensis extract and a Cynara cardunculus var sylvestris extract.

6. Compositions according to claim 1, further comprising extracts of Olea europaea, Berberis aristata, Vitis virufera, Cyclanthera pedata, Silybum marianum and/or carotenoids.
